(19) United States
(12) Patent Application Publication
(10) Pub. No.: US 2018/0242907 A1
(43) Pub. Date: Aug. 30, 2018

(54) DETERMINING METABOLIC PARAMETERS USING WEARABLES

(71) Applicant: KONINKLIJKE PHILIPS N.V., EINDHOVEN (NL)

(72) Inventor: Alberto Giovanni BONOMI, Eindhoven (NL)

(21) Appl. No.: 15/748,029

(22) PCT Filed: Aug. 10, 2016
(86) PCT No.: PCT/IB2016/054811

§ 371 Date: Jan. 26, 2018

Related U.S. Application Data
(60) Provisional application No. 62/203,103, filed on Aug. 10, 2015.

(51) Int. Cl. A61B 5/00; A61B 5/0205; G01N 33/00

(52) U.S. Cl. CPC A61B 5/4866; A61B 5/02055; A61B 5/01; G01N 33/0073; A61B 2560/0242; A61B 5/0002

(57) ABSTRACT

Methods and systems utilize combinations of gaseous concentration, contextual and location information provided by environmental sensors with physiological data provided by wearable sensors to personalize the parameters used in computational models for estimating metabolic parameters. This personalization allows for parameter estimates that better account for the subject-dependent nature of the relationship between heart rate and various metabolic features.

### FIELD OF THE INVENTION

The invention relates generally to methods and systems for determining metabolic parameters, and more specifically to the use of environmental measurements and wearable sensor data to determine metabolic parameters.

### BACKGROUND OF THE INVENTION

Monitoring human metabolic parameters is an attractive feature for systems relating to personal health and chronic disease management. Information on energy expenditure, resting metabolic rate, and cardio-respiratory fitness can be used in innovative coaching programs for weight management, fitness improvement, pregnancy management and chronic disease management. Coaching services may also use metabolic parameters to enhance the physiological response to a specific intervention program so as to enhance the achievement of desired health benefits.

Human metabolic parameters can be estimated using body motion data and physiological data. Energy expenditure and cardio-respiratory fitness can be estimated using wearable sensor data such as heart rate (HR), body acceleration, or other physiological parameters like galvanic skin response (GSR), respiration rate (RR), and body temperature. For example, HR shows a strong and linear relationship with energy expenditure and oxygen uptake (VO₂). However, the characteristics of such a relationship vary among individuals according to parameters such as age, gender, cardio-respiratory fitness, etc.

Several approaches have been proposed to model the person-dependent aspects of the relationship between HR and energy expenditure, including the use of a step protocol or laboratory cycling protocols to characterize the slope coefficient in the relationship. However, these calibration procedures often require laboratory equipment to measure respiratory gas concentrations (e.g., VO₂ and VCO₂).

Similarly, cardio-respiratory fitness can be represented by the HR measured during a specific aerobic task. It has been proposed to determine the relationship between metabolic load and physiological load during physical activity to estimate VO₂max as an index of cardio-respiratory fitness. Again, however, these calibration procedures typically require laboratory equipment to measure respiratory gas concentrations.

Devices used to monitor atmospheric parameters in indoor and outdoor environments typically include multiple sensors such as temperature, humidity, pressure, CO₂ and noise sensors to determine air quality. Data from such a multi-sensor system may be used to gather data from a home environment to assess energy expenditure by combining data on CO₂ concentration with contextual information. However, such systems cannot adequately monitor activities carried outside of an enclosed space and cannot fully evaluate a subject's performance

Accordingly, there is a need for methods and systems that can better utilize physiological and environmental sensor data to estimate the metabolic parameters of one or more subjects.

### SUMMARY OF THE INVENTION

This summary is provided to introduce a selection of concepts in a simplified form that are further described below in the Detailed Description section. This summary is not intended to identify key features or essential features of the claimed subject matter, nor is it intended to be used as an aid in determining the scope of the claimed subject matter.

Embodiments of the present invention provide methods and systems that utilize combinations of contextual and location information provided by environmental sensors with physiological data provided by wearable sensors to personalize the parameters used in computational models for estimating metabolic parameters. This personalization allows for parameter estimates that better account for the subject-dependent nature of the relationship between heart rate and various metabolic features.

In one aspect, embodiments of the present invention relate to a system for estimating metabolic parameters. The system includes a computing unit in communication with a source of environmental sensor data and a source of physiological data. The source of environmental sensor data provides at least one measurement of a gas concentration in an interior space when at least one person is present. The source of physiological data provides at least one physiological measurement concerning a person that is roughly contemporaneous with the at least one environmental measurement. The computing unit computes at least one metabolic parameter associated with the person utilizing, at least in part, the at least one gas concentration measurement and the at least one physiological measurement.

In one embodiment the source of environmental sensor data is a carbon dioxide sensor, an oxygen sensor, both, etc. In one embodiment the source of physiological data is at least one of a cardiometer, an accelerometer, a skin conductance sensor, a respiration rate sensor, and a thermometer. In one embodiment, at least one of the source of physiological data, the source of environmental data, and the computing unit is contained in a wearable device.

In one embodiment the at least one physiological measurement is selected from the group consisting of heart rate, body movement, respiration rate, and body temperature. In one embodiment the at least one environmental measurement is selected from the group consisting of ambient carbon dioxide and ambient oxygen. In one embodiment the at least one metabolic parameter is selected from the group consisting of resting metabolic rate, muscle mass, body composition, energy expenditure and cardio-respiratory fitness.

In one embodiment the computing unit comprises a processing unit configured to personalize a prediction equation using the at least one environmental measurement and apply the personalized prediction equation to the at least one physiological measurement. The processing unit may be further configured to classify the activity of the person based on the at least one physiological measurement. The processing unit may be further configured to determine the respiratory quotient of the person utilizing the classified activity and the at least one environmental measurement.

In another aspect, embodiments of the present invention relate to a method for estimating metabolic parameters. The method includes receiving, at a computing unit, at least one environmental measurement concerning a gas concentration in an interior space from a source of environmental sensor data; receiving, at the computing unit, at least one physiological measurement concerning a person from a source of physiological data; and computing, at the computing unit, at least one metabolic parameter associated with the person utilizing, at least in part, the at least one environmental measurement and the at least one physiological measurement, wherein the at least one environmental measurement is roughly contemporaneous with the presence of the person in the interior space and wherein the at least one physiological measurement concerning the person is roughly contemporaneous with the at least one environmental measurement.

In one embodiment the source of environmental sensor data is at least one of a carbon dioxide sensor and an oxygen sensor. In one embodiment the source of physiological data is at least one of a cardiometer, an accelerometer, a skin conductance sensor, a respiration rate sensor, and a thermometer. In one embodiment at least one of the source of physiological data, the source of environmental data, and the computing unit is contained in a wearable device.

In one embodiment, the at least one physiological measurement is selected from the group consisting of heart rate, body movement, respiration rate, and body temperature. In one embodiment the at least one environmental measurement is selected from the group consisting of ambient carbon dioxide and ambient oxygen. In one embodiment the at least one metabolic parameter is selected from the group consisting of resting metabolic rate, muscle mass, body composition, energy expenditure, and cardio-respiratory fitness.

In one embodiment, the method further includes personalizing, using the computing unit, a prediction equation using the at least one environmental measurement; and applying, using the computing unit, the personalized prediction equation to the at least one physiological measurement. The method may further include classifying, using the computing unit, the activity of the person based on the at least one physiological measurement. The method may further include determining, using the computing unit, the respiratory quotient of the person utilizing the classified activity and the at least one environmental measurement.

These and other features and advantages, which characterize the present non-limiting embodiments, will be apparent from a reading of the following detailed description and a review of the associated drawings. It is to be understood that both the foregoing general description and the following detailed description are explanatory only and are not restrictive of the non-limiting embodiments as claimed.

### BRIEF DESCRIPTION OF DRAWINGS

Non-limiting and non-exhaustive embodiments are described with reference to the following figures in which:

FIG. 1 is a diagram of one embodiment of a system for determining metabolic parameters in accord with the present invention; and

FIG. 2 is a flowchart of a method for estimating metabolic parameters in accord with the present invention.

In the drawings, like reference characters generally refer to corresponding parts throughout the different views. The drawings are not necessarily to scale, emphasis instead being placed on the principles and concepts of operation.

### DETAILED DESCRIPTION OF THE INVENTION

Various embodiments are described more fully below with reference to the accompanying drawings, which form a part hereof, and which show specific exemplary embodiments. However, embodiments may be implemented in many different forms and should not be construed as limited to the embodiments set forth herein; rather, these embodiments are provided so that this disclosure will be thorough and complete, and will fully convey the scope of the embodiments to those skilled in the art. Embodiments may be practiced as methods, systems or devices. Accordingly, embodiments may take the form of a hardware implementation, an entirely software implementation or an implementation combining software and hardware aspects. The following detailed description is, therefore, not to be taken in a limiting sense.

Reference in the specification to “one embodiment” or to “an embodiment” means that a particular feature, structure, or characteristic described in connection with the embodiments is included in at least one embodiment of the invention. The appearances of the phrase “in one embodiment” in various places in the specification are not necessarily all referring to the same embodiment.

Some portions of the description that follow are presented in terms of symbolic representations of operations on non-transient signals stored within a computer memory. These descriptions and representations are the means used by those skilled in the data processing arts to most effectively convey the substance of their work to others skilled in the art. Such operations typically require physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices.

Certain aspects of the present invention include process steps and instructions that could be embodied in software, firmware or hardware, and when embodied in software, could be downloaded to reside on and be operated from different platforms used by a variety of operating systems.

The present invention also relates to an apparatus for performing the operations herein. This apparatus may be specially constructed for the required purposes, or it may comprise a general purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability.

The processes and displays presented herein are not inherently related to any particular computer or other apparatus. Various general purpose systems may also be used with programs in accordance with the teachings herein, or it may prove convenient to construct more specialized apparatus to perform the required method steps. The required structure for a variety of these systems will appear from the description below. In addition, the present invention is not described with reference to any particular programming language. It will be appreciated that a variety of programming languages may be used to implement the teachings of the present invention as described herein, and any references below to specific languages are provided for disclosure of enablement and best mode of the present invention.

In addition, the language used in the specification has been principally selected for readability and instructional purposes, and may not have been selected to delineate or circumscribe the inventive subject matter. Accordingly, the disclosure of the present invention is intended to be illustrative, but not limiting, of the scope of the invention, which is set forth in the claims.

Embodiments of the present invention permit the accurate and unobtrusive assessment of metabolic parameters of users such as total- and activity-energy expenditure, cardio-respiratory fitness and resting metabolic rate using wearable sensors data and personalized prediction equations. The system combines contextual and location information of the user as provided by a wearable sensor (such as heart rate, acceleration, etc.) and an environmental sensor measuring air quality parameters (such as carbon dioxide concentration, air temperature, humidity, noise, pressure, etc.) in indoor spaces. This combination improves the level of personalization of computational methods to accurately assess metabolic parameters such as energy expenditure, resting metabolic rate, and cardio-respiratory fitness

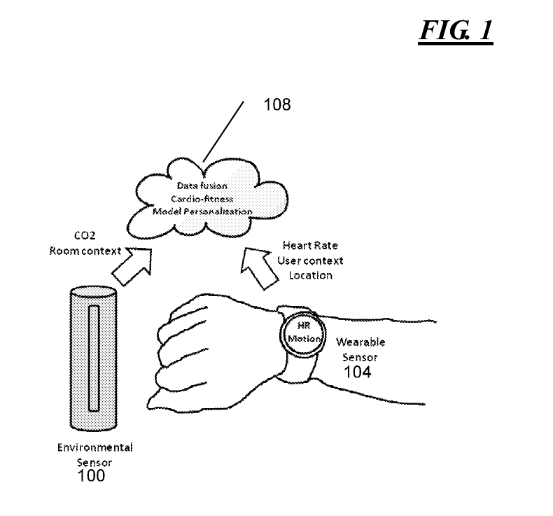

FIG. 1 presents one embodiment of a system for estimating metabolic parameters in accord with the present invention. An environmental sensor 100 provides measurements of environmental factors, such as a gas concentration, in a monitored space. The environmental sensor 100 is depicted as a discrete, standalone appliance, although it may take a variety of configurations and may, in some embodiments, be integrated into the wearable sensor 104 and/or the computing unit 108, may consist of a set of distributed sensors, etc. The environmental sensor 100 may take a variety of forms as well, including but not limited to a weather station, a carbon dioxide sensor, a barometer, a humidity sensor, a combination of any of the foregoing, etc. The gas concentrations monitored will typically be relevant to the estimation of metabolic parameters, as discussed in greater detail below, such as ambient carbon dioxide, ambient oxygen, etc., although other gases and environmental factors may be monitored as well.

A variety of wearable sensors 104 may be used to provide measurements of physiological data for one or more people present in the space monitored by the environmental sensor 100. The wearable sensor 104 may take a variety of forms (wrist-worn, chest-worn, carried, etc.), and may include one or more sensors such as a cardiometer, an accelerometer, a galvanometer, a thermometer, a GPS sensor in a smartphone, etc.

The computing unit 108 may take a variety of forms, such as a local desktop or laptop computer, a set top box, an app executing on a smartphone, a tablet, a “next unit of computing” (NUC), a wireless speaker, or a remote server computer in communication with one or more of the foregoing devices, etc., but regardless of particular configuration includes sufficient computing capacity to execute the methods described below.

As mentioned above, in various embodiments the three discrete components (environmental sensor 100, wearable sensor 104, and computing unit 108) may be integrated and reorganized into a single component, two components (e.g., combined environmental sensor/wearable sensor and computing unit; combined environmental sensor/computing unit and wearable sensor, etc.), or greater than three components (e.g., multiple environmental sensors, multiple wearables, etc.).

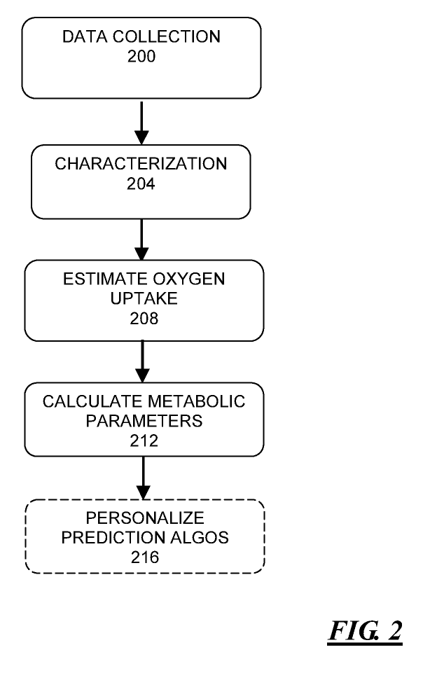

FIG. 2 presents a flowchart of a method for estimating metabolic parameters utilizing the system of FIG. 1 or an equivalent system having sources of environmental data and physiological data.

In overview, the process begins with the collection of relevant environmental and physical data (Step 200). The collected data is used to make one or more characterizations about the environment and/or the person being monitored, e.g., that the person is running on a treadmill in a room (Step 204). The characterizations can be used with the collected data to estimate the user's oxygen uptake (Step 208), to calculate various metabolic parameters (Step 212) and to optionally personalize the metabolic parameter prediction models (Step 216) applied to the oxygen uptake value to determine the various metabolic parameters.

The data collection process (Step 200) is conducted so that the physiological data and environmental data are collected substantially contemporaneously. In some embodiments the collection is synchronous; in other embodiments, the collection is substantially simultaneous. The collection process can be active, e.g., with a computing unit polling one or more sensors, passive, e.g., with a computing unit accessing one or more log files or receiving periodic transmissions from a sensor, or some combination of the two depending on, e.g., the nature of the sensor, the type of the data, the time of day, etc. This collection process may occur on demand over a course of minutes or it may be an ongoing process spanning durations varying from hours to months.

The environmental data may include, but is not limited to, temperature, atmospheric pressure, humidity, volume, noise level, carbon dioxide concentration, other gas concentrations, etc., which may be used in the determination of metabolic parameters and activities occurring in the enclosed space containing the sensors as discussed below. The physiological data may include heart rate, galvanic skin response, body temperature, as well as biomechanical data such as accelerometer data or positioning data.

The process of characterization, a.k.a. “contextualization” (Step 204) has been described in U.S. Provisional Appl. 62/190,297, filed on Jul. 9, 2015, which is hereby incorporated by reference as if set forth in its entirety herein. Embodiments of the present invention draw one or more inferences concerning the context of the room and/or the monitored user; for example, whether the room is empty or whether there are one or more persons in the space, preferably in proximity to a sensor in that space. Other possible inferences concern the reliability of the environmental data, whether a plurality of subjects are present in the interior space, whether the subject(s) is/are engaged in activity, the nature of the activity that the subject(s) is/are engaged in, etc.

The numbers of users in the interior space can be determined using environmental sensors data. For example, a microphone may detect one or more voices and infer the presence of one or more users. Measurements of carbon dioxide or other gas concentrations can also be used to infer the presence of one or more users, as can measurements of ambient room temperature or the use of a pyrometer to identify person-sized sources of ambient heat.

The activity performed by a particular user can be determined using physiological data. For example, accelerometer data can be used to identify when a user is sedentary or performing some activity such as walking or running. Cardiometer and skin conductance data can be used to determine the user's heart rate and, indirectly, whether the user is active or sedentary.

The fusion of several kinds of sensor data may be used to perform a more refined classification of the user's activities. While an initial classification may simply distinguish between active and sedentary states, access to location state, calendar data, social media data, T.V. schedule data, etc., may permit the system to determine that a user is not just sedentary, but also watching television. Similarly a user may be determined to be exercising based on GPS data indicating their location in a gym, vacuuming due to an increase in the electricity drawn in the user's home, or performing an intervention program based on schedule data taken from the user's electronic calendar.

In some embodiments, contextual awareness may be substituted or augmented by user input. Information on whether a specific activity is being carried out in the room can be provided by the user, as well as information on the status of the environment such as whether doors and windows are open or closed.

In some embodiments, the robustness of the computational method is improved by accounting for environmental influences on the physiological data used to estimate energy expenditure such as temperature and humidity.

The estimation of oxygen uptake (Step 208) takes into account the contextual information determined in the characterization step (Step 204), as well as direct measurements of VCO₂ present in the environmental data, estimates of respiratory quotient, and other data gathered during the collection phase (Step 200). Techniques for such estimation are also discussed in U.S. Provisional Application No. 62/190,297, incorporated by reference above, which also describes how such measurements may be adjusted to take into account the nature of the activity performed by the user.

In one embodiment, the oxygen uptake (VO₂) is determined from measurements of VCO₂ and estimates of the user's respiratory quotient (RQ), i.e., the ratio between VCO₂ and VO₂, that take into account the type of activity undertaken by the user given the contextual information provided by a wearable sensor. Given a direct measurement of VCO₂ in the enclosed space as well as an estimate of RQ given the detected activity, it is trivial to determine the associated value of VO₂.

Data on VO₂ uptake is combined with wearable sensor data and contextual information to compute various metabolic parameters (Step 212).

VO₂ data recorded during sedentary periods is used to establish the resting metabolic rate (RMR), which is a key parameter for evaluating total energy expenditure during the day. Information on resting VO₂ and RMR can also be consequently used to estimate other metabolic parameters such as body composition.

If the activity classifier indicates that the user is undertaking activity of moderate or vigorous intensity in the measured interior space, then various metabolic parameters may be determined utilizing the ratio between the estimated VO₂ uptake and various physiological parameters. For example, the oxygen pulse factor (i.e., the ratio of VO₂ to HR) can be used to determine an individual's VO₂max, i.e., the reference index for fitness level; and other parameters indicating physical performance such as anaerobic threshold and fatigue by monitoring trends in oxygen pulse over time.

Data on VO₂ uptake can also be combined with wearable sensor data and contextual information to personalize the models used to estimate metabolic parameters such as energy expenditure and cardio-respiratory fitness (Step 216). Model personalization is achieved by characterizing the relationship between VO₂ and the target physiological parameter (e.g., HR).

For example, the physical fitness of an individual user may be ascertained from a variety of sources, including manual entry, the monitoring of physical performance by a wearable device, and/or an evaluation using an untuned model. With an evaluation of physical fitness, the parameters of the model can then be adjusted to account for the user's fitness level.

For example, the slope and intercept in a linear prediction model comparing the ratio of VO₂/HR to VO₂max can be altered for different users or for the same user over time due to changes in a particular user's physical condition, disease progression and medication intake. Stress, sleep quality, overtraining, and activity recovery are some of the many physiological factors that may alter the relationship between VO₂/HR and VO₂max. Additionally, changes in VO₂/HR over time are used to determine variations in VO₂max for a specific user condition determined by contextual data.

Information on energy expenditure, cardio-respiratory fitness, and resting metabolic rate can be integrated in innovative coaching programs for weight management, fitness improvement, pregnancy management and chronic disease management. Coaching services may use metabolic data to personalize and enhance the physiological response to a specific intervention program so to maximize the desired health benefit.

Embodiments of the present disclosure, for example, are described above with reference to block diagrams and/or operational illustrations of methods, systems, and computer program products according to embodiments of the present disclosure. The functions/acts noted in the blocks may occur out of the order as shown in any flowchart. For example, two blocks shown in succession may in fact be executed substantially concurrent or the blocks may sometimes be executed in the reverse order, depending upon the functionality/acts involved. Additionally, not all of the blocks shown in any flowchart need to be performed and/or executed. For example, if a given flowchart has five blocks containing functions/acts, it may be the case that only three of the five blocks are performed and/or executed. In this example, any of the three of the five blocks may be performed and/or executed.

The description and illustration of one or more embodiments provided in this application are not intended to limit or restrict the scope of the present disclosure as claimed in any way. The embodiments, examples, and details provided in this application are considered sufficient to convey possession and enable others to make and use the best mode of the claimed embodiments. The claimed embodiments should not be construed as being limited to any embodiment, example, or detail provided in this application. Regardless of whether shown and described in combination or separately, the various features (both structural and methodological) are intended to be selectively included or omitted to produce an embodiment with a particular set of features. Having been provided with the description and illustration of the present application, one skilled in the art may envision variations, modifications, and alternate embodiments falling within the spirit of the broader aspects of the general inventive concept embodied in this application that do not depart from the broader scope of the claimed embodiments.

1. A system for estimating metabolic parameters, the system comprising:
a computing unit in communication with:
a source of environmental sensor data providing at least one measurement of a gas concentration in an interior space; and
a source of physiological data providing at least one physiological measurement concerning a person,
wherein the source of environmental sensor data is used to obtain at least one gas concentration measurement concerning the interior space when at least one person is present,
the source of physiological data is used to obtain at least one physiological measurement concerning the person that is substantially contemporaneous with the at least one environmental measurement, and
the computing unit is used to compute at least one metabolic parameter associated with the person utilizing, at least in part, the at least one gas concentration measurement and the at least one physiological measurement.

2. The system of claim 1 wherein the source of environmental sensor data is at least one of a carbon dioxide sensor and an oxygen sensor.

3. The system of claim 1 wherein the source of physiological data is at least one of a cardiometer, an accelerometer, a skin conductance sensor, a respiration rate sensor, and a thermometer.

4. The system of claim 1 wherein at least one of the source of physiological data, the source of environmental data, and the computing unit is contained in a wearable device.

5. The system of claim 1 wherein the at least one physiological measurement is selected from the group consisting of heart rate, body movement, respiration rate, and body temperature.

6. The system of claim 1 wherein the at least one environmental measurement is selected from the group consisting of ambient carbon dioxide and ambient oxygen.

7. The system of claim 1 wherein the at least one metabolic parameter is selected from the group consisting of resting metabolic rate, muscle mass, body composition, energy expenditure and cardio-respiratory fitness.

8. The system of claim 1 wherein the computing unit comprises a processing unit configured to:
personalize a prediction equation using the at least one environmental measurement; and
apply the personalized prediction equation to the at least one physiological measurement.

9. The system of claim 8 wherein the processing unit is further configured to classify the activity of the person based on the at least one physiological measurement.

10. The system of claim 9 wherein the processing unit is further configured to determine the respiratory quotient of the person utilizing the classified activity and the at least one environmental measurement.

11. A method for estimating metabolic parameters, the method comprising:
receiving, at a computing unit, at least one environmental measurement concerning a gas concentration in an interior space from a source of environmental sensor data;
receiving, at the computing unit, at least one physiological measurement concerning a person from a source of physiological data; and
computing, at the computing unit, at least one metabolic parameter associated with the person utilizing, at least in part, the at least one environmental measurement and the at least one physiological measurement,
wherein the at least one environmental measurement is substantially contemporaneous with the presence of the person in the interior space, and
wherein the at least one physiological measurement concerning the person is substantially contemporaneous with the at least one environmental measurement.

12. The method of claim 11 wherein the source of environmental sensor data is at least one of a carbon dioxide sensor and an oxygen sensor.

13. The method of claim 11 wherein the source of physiological data is at least one of a cardiometer, an accelerometer, a skin conductance sensor, a respiration rate sensor, and a thermometer.

14. The method of claim 11 wherein at least one of the source of physiological data, the source of environmental data, and the computing unit is contained in a wearable device.

15. The method of claim 11 wherein the at least one physiological measurement is selected from the group consisting of heart rate, body movement, respiration rate, and body temperature.

16. The method of claim 11 wherein the at least one environmental measurement is selected from the group consisting of ambient carbon dioxide and ambient oxygen.

17. The method of claim 11 wherein the at least one metabolic parameter is selected from the group consisting of resting metabolic rate, muscle mass, body composition, energy expenditure and cardio-respiratory fitness.

18. The method of claim 11 further comprising:
personalizing, using the computing unit, a prediction equation using the at least one environmental measurement; and
applying, using the computing unit, the personalized prediction equation to the at least one physiological measurement.

19. The method of claim 18 further comprising classifying, using the computing unit, the activity of the person based on the at least one physiological measurement.

20. The method of claim 19 further comprising determining, using the computing unit, the respiratory quotient of the person utilizing the classified activity and the at least one environmental measurement.
